(19) United States
(12) Patent Application Publication
(10) Pub. No.: US 2021/0236569 A1
(43) Pub. Date: Aug. 5, 2021

(54) BIOACTIVE FUNGAL COMPOUNDS PRODUCED BY SOLID-STATE FERMENTATION

(71) Applicant: KAIROS BIOMEDICAL, INC., Kailua, HI (US)

(72) Inventor: Gary L. MILLS, Kailua, HI (US)

(21) Appl. No.: 17/049,938

(22) PCT Filed: Apr. 22, 2019
(86) PCT No.: PCT/US2019/028500

§ 371 Date: Oct. 22, 2020

Related U.S. Application Data
(60) Provisional application No. 62/663,878, filed on Apr. 27, 2018.

(51) Int. Cl. A61K 36/07; A61K 36/062; A61P 29/00; C12P 19/04; A23L 31/00; A23L 33/17; A23L 33/115; A23L 33/125; A23L 33/14; A23L 33/16; A23K 10/30; A23K 20/163; A23K 20/147; A23K 20/158; A23K 20/24

(52) U.S. Cl. CPC A61K 36/07; A23V 2002/00; A61P 29/00; C12P 19/04; A23L 31/00; A23L 33/17; A23L 33/115; A23L 33/125; A23L 33/14; A23L 33/16; A23K 10/30; A23K 20/163; A23K 20/147; A23K 20/158; A23K 20/24; A61K 2236/51; A61K 2236/19; A61K 36/062

(57) ABSTRACT

Two unique bioactive compounds, designated K1 and K2, were extracted from a solid substrate that had been inoculated with filamentous fungi and incubated for an extended time. The compounds ranged from 20% to 30% of starting dry weight after concentration and the full ¹H NMR spectra showed a complex mixture of aromatic, polysaccharides/sugars, and aliphatic amino, lipid and organic acid components. These bioactive fungal extracts exhibited strong immune activating properties.

### PRIORITY AND CROSS-REFERENCE TO RELATED APPLICATIONS

This application claims the benefit of U.S. Provisional Application 62/663,878, filed on Apr. 27, 2018, which is hereby incorporated by reference in its entirety.

### BACKGROUND

### Field

The present disclosure relates to a process for producing and obtaining biologically active fungal compounds using solid-state fermentation.

### Description of the Related Art

There is an enormous body of evidence that supports the notion that many chronic diseases can be prevented, or at least substantially delayed. Thousands of phytochemicals are known to protect against disease. Often grouped with these are medicinal fungi with medicinal mushrooms being among the examples of foods that contain disease fighting ingredients. The practice of using mushrooms as “medical foods” is found in the traditions of many cultures. The early herbalist probably was more interested in the medicinal properties of mushrooms than using them as food.

### SUMMARY

In some embodiments, a solid-state fermentation method for producing a dried product comprising bioactive fungal compounds is provided. In some embodiments, the method comprises inoculating a solid substrate with one or more mushroom mycelium cultures, incubating the inoculated solid substrate for a period of about 10-100 days under conditions selected to promote colonization and enzymatic hydrolysis of the solid substrate to produce a hydrolysate, extracting the hydrolysate in water at a temperature of about 60° C.-100° C. with agitation, treating the extract with amylase, clarifying the extract to remove undigested material, and drying the clarified extract to yield a dried product comprising bioactive fungal compounds. In some embodiments of the method, the solid substrate comprises a hydrated cooked grain. In some embodiments of the method, the hydrated cooked grain comprises one or more grains selected from rice, wheat, millet, oats or rye, and wherein the one or more grains are hydrated in water and cooked for about 10-90 minutes at a temperature of about 70° C.-120° C. In some embodiments of the method, the grains are cooked for about 30-60 minutes at 100° C. In some embodiments of the method, the solid substrate further comprises one or more of a carbohydrate, a nitrogen source and a mineral supplement. In some embodiments of the method, the carbohydrate is selected from glucose, sucrose, maltose, malt extract and/or corn steep liquor. In some embodiments of the method, the nitrogen source is selected from peptone, soybean powder, soy grits, whole yeast and/or yeast extract. In some embodiments of the method, the mineral supplement is selected from phosphates, calcium, magnesium, iron, and/or sulfates. In some embodiments of the method, the solid substrate is sterilized before inoculation. In some embodiments of the method, the solid substrate is sterilized by autoclaving for about 30-120 minutes at a temperature of about 100° C.-200° C. at a pressure of about 10-30 psi. In some embodiments of the method, the solid substrate is sterilized by autoclaving for about 60 minutes at a temperature of about 121° C. at a pressure of about 15-18 psi. In some embodiments of the method, the one or more mushroom mycelium cultures comprise a fungal class selected from Ascomycetes or Basidiomycetes. In some embodiments of the method, the one or more mushroom mycelium cultures are Morchella rufobrunnea from the Ascomycetes class. In some embodiments of the method, the one or more mushroom mycelium cultures are from the Basidiomycetes class, selected from Coriolus versicolor, Lentinula edodes or Schizophyllum commune. In some embodiments of the method, the incubation conditions comprise incubating in the dark for 30-60 days. In some embodiments of the method, the incubation conditions comprise incubating at a temperature of about 10° C.-40° C. In some embodiments of the method, the incubation temperature is about 16° C.-21° C. In some embodiments of the method, the incubation conditions comprise incubating at a relative humidity of about 50%-100%. In some embodiments of the method, the incubation relative humidity is about 70%. In some embodiments of the method, drying comprises freeze-drying (lyophilization), spray-drying, heating, drying under nitrogen and/or combinations thereof. In some embodiments, the method further comprises formulating the dried product into a form suitable for delivery as a dietary supplement, nutraceutical, medical food or animal feedstuff.

In some embodiments, a dried product comprising bioactive fungal compounds produced by any of the methods disclosed herein is provided. In some embodiments of the dried product produced by any of the methods disclosed herein, the one or more mushroom mycelium cultures comprise different strains of Lentinula edodes. In some embodiments of the dried product produced by any of the methods disclosed herein, the solid substrate comprises of hydrated cooked rice, wheat or rye. In some embodiments of the dried product produced by any of the methods disclosed herein, the incubation period comprises 60 days. In some embodiments of the dried product produced by any of the methods disclosed herein, the dried product is designated K1, and is characterized by the following properties—about 81.5% by weight carbohydrates; about 3.4% by weight protein; about 0.7% lipid by weight; about 14.4% by weight of other components; and immunomodulatory bioactivity. In some embodiments, the dried product is further characterized by exhibiting 3 retention time peaks at about 10.5, 11.2, and 24.0 by HPLC of a de-lipidized dried product sample run on an Agilent Hi-Plex Na column eluted with water at 0.3 ml/min at 85° C. and 25 bar pressure, by and UV detection (280 nm). In some embodiments of the dried product, the immunomodulatory bioactivity comprises at least one of the following—activation of one or more immune cells selected from NK cells, NKT cells, T-lymphocytes, Non-T and Non-NK lymphocytes, or monocytes; and activation of pro-inflammatory cytokines/chemokines, or anti-inflammatory cytokines/chemokines, or both pro- and anti-inflammatory cytokines/chemokines.

In some embodiments, a dried product comprising bioactive fungal compounds produced by any of the methods disclosed herein is provided. In some embodiments of the dried product produced by any of the methods disclosed herein, the one or more mushroom mycelium cultures comprise a mixture of Coriolus versicolor, Morchella rufobrunnea, and Schizophyllum commune. In some embodiments of the dried product produced by any of the methods disclosed herein, the solid substrate comprises supplemented with soy grits, whole yeast, sucrose and gypsum hydrated cooked wheat; and the incubation period comprises 35-45 days. In some embodiments of the dried product produced by any of the methods disclosed herein, the dried product is designated K2 and is characterized by the following properties—about 66.8% by weight carbohydrates; about 4.1% by weight protein; about 2.7% lipid by weight; about 26.4% by weight of other components; and immunomodulatory bioactivity. In some embodiments, the dried product is further characterized by exhibiting 2 retention time peaks at about 14.3 and 15.2 by HPLC of a de-lipidized dried product sample run on an Agilent Hi-Plex Na column eluted with water at 0.3 ml/min at 85° C. and 25 bar pressure, by and UV detection (280 nm). In some embodiments of the dried product, the immunomodulatory bioactivity comprises activation of one or more cells selected from NK cells, B-lymphocytes, dendritic cells, stem cells or monocytes.

In some embodiments, a dried product comprising bioactive fungal compounds is provided. In some embodiments, the dried product is characterized by the following properties—60%-90% by weight carbohydrates, comprising polysaccharides including α(1-4) glucans and β(1-3) glucans as determined by 1H and 13C NMR spectrum, the polysaccharides having a median molecular weight of about 10,000 Da; 3%-5% by weight protein; 0.1%-3% by weight lipid; 2 or 3 retention time peaks by HPLC of de-lipidized sample on Agilent Hi-Plex Na column; and immunomodulatory bioactivity.

In some embodiments, the dried product is in a form suitable for delivery as a dietary supplement, nutraceutical, medical food or animal feedstuff.

### DETAILED DESCRIPTION

Medicinal fungi have been the subject of focused research. Medicinal fungi are claimed to exhibit antiviral, antibiotic, anti-inflammatory, antidiabetic, and antitumor activities. Many are known to enhance the immune system and are regarded as biological response modifiers and immunomodulators. The bioactive components from fungi include polysaccharides, terpenes, phenols, amino acids, lectins, statins, nucleotides, sterols and glucosylceramides. Numerous preparations made from medicinal fungi are produced in the form of capsules, ampules, extracts or teas and are sold as dietary supplements or nutraceuticals. In Japan, the polysaccharides lentinan, krestin (PSK), grifolan and schizophyllan obtained from the respective fungi Lentinula edodes, Coriolus versicolor, Grifolia frondosa, and Schizophyllum commune have been registered as anti-cancer drugs since mid-1980s.

Medicinal fungi are usually higher fungi, members of the Ascomycete or Basidiomycete class, and are a rich source of biologically active polysaccharides that are used as adaptogens and immunostimulators that achieve their effect through the activation of various defensive immune responses. These biologically active polysaccharides can be obtained from the mushroom fruit body or mycelium obtained from cultivation using submerged liquid fermentation or solid-state fermentation. The cultivation of mushrooms for fruiting bodies is a long-term process needing many weeks of incubation in a vegetative phase and often many more weeks in controlled environmental setting for fruiting body formation. Mycelium submerged liquid fermentation is a known process for growing medicinal fungi but production is at a relatively high cost utilizing expensive bioreactors. An alternative method is cultivating mycelium using solid-state fermentation which is much less costly. A criticism has been leveled that this method of cultivation has high levels of starch related a glucans and lower quantities of bioactive β glucans.

Recently, a unique bioactive compound was produced during submerged liquid fermentation of Lentinula U.S. Pat. No. 5,756,318 to Kosuna. The compound produced was named AHCC, active hexose correlated compound, that is available as a dietary supplement. The complex compound contains a mixture of polysaccharides, amino acids, lipids and minerals. The predominant oligosaccharides total 74% of the total dry weight. Of these, nearly 20% are partially acetylated a 1-4 glucans, which are believed to be the active compounds of AHCC. Supplementation studies with AHCC have demonstrated positive effects on immune function in human and animal models. These include: decreased tumor formation, increased resistance to viral and bacterial infection, enhanced natural killer cell activation, altered cytokine production, increased dendritic cell function, increased T-cell proliferation, increased nitric oxide release by peritoneal cells, and antioxidant and anti-inflammatory effects.

In some embodiments, the present disclosure relates to a process for producing and obtaining biologically active fungal nutraceutical compounds from the cultivation of filamentous fungi and biologically active fungal nutraceutical compounds so obtained. In some embodiments, the present disclosure relates to a process for producing and obtaining biologically active fungal nutraceutical compounds from the cultivation of filamentous fungi by way of solid-state fermentation and biologically active fungal nutraceutical compounds so obtained. In some embodiments, the process allows for increased cost-effective yields of complex biologically active fungal nutraceutical compounds. In some embodiments, a key component of these biologically active fungal nutraceutical compounds is their high carbohydrate content, primarily α and β glucans, and these fungal polysaccharides have been shown to be important in stimulating the innate immune system and activation of effector cells.

In some embodiments, the disclosure is related at production of high yielding biologically active compounds isolated from the cultivation of medicinal filamentous fungi utilizing solid-state fermentation. In some embodiments, the medicinal fungi grown are members of the Ascomycete and Basidiomycete class of fungi, while the solid fermentation media is hydrated grain with or without additional supplementation of carbohydrates, nitrogen, vitamins and inorganic components.

In some embodiments, a cultivation of the medicinal fungi for a duration of time allows for the enzymatic breakdown of the substrate through the process of fermentation with production of secondary metabolites and the formation of functional biologically active compounds.

Thus, in some embodiments, the present disclosure provides a method to obtain high yields of bioactive components that include both acetylated a 1-4 glucans as well as 1-3 glucans.

In some embodiments, the bioactive components may be used as a stand-alone dietary supplement. In some embodiments, the bioactive components are formulated with other compounds into nutraceuticals or possibly medical foods. Alternatively, in some embodiments, the bioactive compounds can be mixed with feed material for use in poultry, swine/beef industries or feed for aquaculture, which may help to reduce the use of antibiotics in the food industries.

### Solid-State Fermentation Methods

In some embodiments, provided herein is a method of harvesting a fungal material impregnated solid substrate that is subsequently hot water extracted and treated enzymatically to further breakdown the fungal material to obtain a extracted slurry. In some embodiments, the enzymatically treated hot water extracted slurry is centrifuged and/or filtered to remove larger debris and the supernatant and/or filtrate is concentrated into a final dried product.

In some embodiments, a solid-state fermentation method is provided for producing a dried product or extract comprising bioactive fungal compounds. In some embodiments, the dried product comprising bioactive fungal compounds comprises biologically active fungal nutraceutical compounds.

In some embodiments, the method comprises inoculating a solid substrate. In some embodiments, the solid substrate is inoculated with one or more mushroom mycelium cultures. In some embodiments, the inoculated solid substrate is incubated for a period of time. In some embodiments, the period of time is about 10 days to about 100 days. In some embodiments, the period of time is about 2 days to about 500 days. In some embodiments, the period of time is about 2, 5, 10, 15, 20, 30, 40, 50, 60, 80, 100, 150, 200, 250, 300, 350, 400, 450, or 500 days, or a value within a range defined by any two of the aforementioned values.

In some embodiments, the inoculated solid substrate is incubated under conditions selected to promote colonization and enzymatic hydrolysis of the solid substrate to produce a hydrolysate.

In some embodiments, the hydrolysate is extracted. In some embodiments, the hydrolysate is extracted in a solvent. In some embodiments, the solvent is water. In some embodiments, the solvent is one or more organic solvents. In some embodiments, the solvent is water and one or more organic solvents. In some embodiments, the hydrolysate is extracted at a temperature of about 60° C. to about 100° C. with agitation. In some embodiments, the temperature is about 40° C. to about 120° C. In some embodiments, the temperature is about 40, 45, 50, 55, 60, 65, 70, 75, 80, 85, 90, 95, 100, 105, 110, 115, or 120° C., or a value within a range defined by any two of the aforementioned values.

Methods for agitation are well known in the art, for example, shaking, hand blending, vortexing, mechanical mixing with stir bar, agitation tanks or paddle mixers.

In some embodiments, the extract is treated with one or more enzymes. In some embodiments, the one or more enzymes include amylase, protease, glucanase or amyloglucanase.

In some embodiments, the extract is clarified after treating with one or enzymes. In some embodiments, the extract is clarified to remove undigested material. Methods for clarification are well known in the art, for example, decanting, filtration, centrifugation, sedimentation, and the like.

In some embodiments, the solid substrate comprises a hydrated cooked grain. In some embodiments, the hydrated cooked grain comprises one or more grains selected from rice, wheat, millet, oats or rye.

In some embodiments, the one or more grains are hydrated in water and cooked for about 10 minutes to about 90 minutes at a temperature of about 70° C. to about 120° C. In some embodiments, the one or more grains are cooked for about 5 to about 180 minutes. In some embodiments, the one or more grains are cooked for about 30 to about 60 minutes. In some embodiments, the one or more grains are cooked for about 5, 10, 20, 40, 60, 80, 100, 120, 140, 160, or 180 minutes, or a value within a range defined by any two of the aforementioned values. In some embodiments, the temperature is about 50° C. to about 140° C. In some embodiments, the temperature is about 100° C. In some embodiments, the temperature is about 50, 55, 60, 65, 70, 75, 80, 85, 90, 95, 100, 105, 110, 115, 120, 125, 130, 135, or 140° C., or a value within a range defined by any two of the aforementioned values.

In some embodiments, the solid substrate further comprises one or more additional components. In some embodiments, the one or more additional components comprises a carbohydrate. In some embodiments, the one or more additional components comprises a nitrogen source. In some embodiments, the one or more additional components comprises a mineral supplement. In some embodiments, the one or more additional components comprises a carbohydrate, and a nitrogen source. In some embodiments, the one or more additional components comprises a carbohydrate, and a mineral supplement. In some embodiments, the one or more additional components comprises a nitrogen source and a mineral supplement. In some embodiments, the one or more additional components comprises a carbohydrate, a nitrogen source and a mineral supplement.

In some embodiments, the carbohydrate is selected from glucose, sucrose, maltose, malt extract and/or corn steep liquor.

In some embodiments, the nitrogen source is selected from peptone, soybean powder, soy grits, whole yeast and/or yeast extract.

In some embodiments, the mineral supplement is selected from phosphates, calcium, magnesium, iron, and/or sulfates

In some embodiments, the solid substrate is sterilized before inoculation. Non-limiting examples of methods of sterilization include heat sterilization by steam, dry heat, autoclaving, tyndallization, and/or radiation sterilization by non-ionizing radiation, and ionizing radiation. In some embodiments, the solid substrate is sterilized by autoclaving. In some embodiments, the solid substrate is sterilized by autoclaving for about 30 minutes to about 120 minutes at a temperature of about 100° C. to about 200° C. at a pressure of about 10 psi to about 30 psi. In some embodiments, the solid substrate is sterilized by autoclaving for about 60 minutes at a temperature of about 121° C. at a pressure of about 15 psi to about 18 psi. In some embodiments, the solid substrate is sterilized for about 15 minutes to about 240 minutes. In some embodiments, the solid substrate is sterilized for about 60 minutes. In some embodiments, the solid substrate is sterilized for about 15, 30, 45, 60, 80, 100, 120, 140, 160, 180, 200, or 240 minutes, or a value within a range defined by any two of the aforementioned values. In some embodiments, the solid substrate is sterilized at a temperature of about 50° C. to about 400° C. In some embodiments, the solid substrate is sterilized at a temperature of about 121° C. In some embodiments, the solid substrate is sterilized at a temperature of about 50, 60, 70, 80, 100, 125, 150, 175, 200, 225, 250, 275, 300, 325, 350, 375, or 400° C., or a value within a range defined by any two of the aforementioned values. In some embodiments, the solid substrate is sterilized at a pressure of about 5 psi to about 60 psi. In some embodiments, the solid substrate is sterilized at a pressure of about 15 psi to about 18 psi. In some embodiments, the solid substrate is sterilized at a pressure of about 13, 13.5, 14, 14.5, 15, 15.5, 16, 16.5, 17, 17.5, 18, 18.5, 19, 19.5, or 20 psi, or a value within a range defined by any two of the aforementioned values. In some embodiments, the solid substrate is sterilized at a pressure of about 5, 10, 15, 20, 25, 30, 35, 40, 45, 50, 55, or 60 psi, or a value within a range defined by any two of the aforementioned values.

In some embodiments, the one or more mushroom mycelium cultures comprise a fungal class selected from Ascomycetes. In some embodiments, the one or more mushroom mycelium cultures comprise a fungal class selected from Basidiomycetes. In some embodiments, the one or more mushroom mycelium cultures comprise a fungal class selected from Ascomycetes or Basidiomycetes. In some embodiments, the one or more mushroom mycelium cultures comprise a fungal class selected from Ascomycetes or Basidiomycetes.

In some embodiments, the one or more mushroom mycelium cultures are Morchella rufobrunnea from the Ascomycetes class. In some embodiments, the one or more mushroom mycelium cultures are from the Basidiomycetes class, selected from Coriolus versicolor, Lentinula edodes or Schizophyllum commune.

In some embodiments, the methods comprise culturing filamentous fungal mycelium of species of Ascomycetes, for example, the edible species of the genus Morchella, or Basidiomycetes that are currently considered edible or used as dietary supplements. The preferred Basidiomycete species, but not limited to, are those shown in Table 1.

TABLE 1

| Mushroom Species | Edible | Dietary Suppl. |
|---|---|---|
| Agaricus blazei | + | + |
| Agrocybe aegerita | + | + |
| Antrodia camphorata | | + |
| Armillariella tabescens | | + |
| Auricularia auricula | + | + |
| Cordyceps sinensis | | + |
| Coriolus(Tremetes) versicolor | | + |
| Flammulina velutipes | + | + |
| Ganoderma lucidum | | + |
| Ganoderma tsugae | | + |
| Grifola frondosa | + | + |
| Hericium erinaceus | + | + |
| Hypsizigus marmoreus | + | + |
| Hypsizigus marmoreus | + | + |
| Inonotus obliquus | | + |
| Lentinula edodes | + | + |
| Pleurotus citrinopileatus | + | + |
| Pleurotus eryngii | + | + |
| Pleurotus ostreatus | + | + |
| Pleurotus sajor-caju | + | + |
| Pholiota nameko | + | + |
| Polyporus umbellatus | | + |
| Poria cocus | | + |
| Schizophyllum commune | | + |
| Tremella fuciformis | + | + |
| Volvariella volvacea | + | + |

In some embodiments, the incubation conditions comprise incubating in the dark for about 30 days to about 60 days. In some embodiments, the incubation conditions comprise incubating for about 10 days to about 180 days. In some embodiments, the incubation conditions comprise incubating for about 10, 20, 30, 40, 50, 60, 70, 80, 90, 100, 110, 120, 230, 140, 150, 160, 170, or 180 days, or a value within a range defined by any two of the aforementioned values.

In some embodiments, the incubation conditions comprise incubating at a temperature of about 10° C. to about 40° C. In some embodiments, the incubation temperature is about 16° C. to about 21° C. In some embodiments, the incubation temperature is about 5° C. to about 50° C. In some embodiments, the incubation temperature is about 5, 10, 15, 20, 25, 30, 35, 40, 45, or 50° C., or a value within a range defined by any two of the aforementioned values.

In some embodiments, the incubation conditions comprise incubating at a relative humidity of about 50% to about 100%. In some embodiments, the incubation relative humidity is about 70%. In some embodiments, the incubation relative humidity is about 5, 10, 15, 20, 25, 30, 35, 40, 45, 50, 55, 60, 65, 70, 75, 80, 85, 90, 95, or 100%, or a value within a range defined by any two of the aforementioned values.

Methods of drying are well known in the art, for example, baking, freeze-drying (lyophilization), spray-drying, heating, drying under nitrogen, drying under vacuum and/or combinations thereof.

### Chemical Characterization of Fungal Products

In some embodiments, the disclosure further comprises chemical characterization of the properties of the dried fungal product. In some embodiments, the resulting yields of the dried product according to the methods of the present disclosure are between about 20% to about 30% of the dry weight of the starting material. In some embodiments, the resulting yields of the dried product are between about 25% to about 65% of the dry weight of the starting material. In some embodiments, the resulting yields of the dried product are about 25, 30, 35, 40, 45, 50, 55, 60, or 65% of the dry weight of the starting material, or a value within a range defined by any two of the aforementioned values.

In some embodiments, the major components of the dried product produced according to the methods of the present disclosure are sugars representing 70%-80% of the total by weight and of these between 9%-25% are a 1-4 glucans and 8%-30% β 1-3 glucans. In some embodiments, the minor components of the dried product produced according to the methods of the present disclosure, are proteins (3%-4%) and lipid (1%-3%) by weight. In some embodiments, the balance weight of the dried product produced according to the methods of the present disclosure is composed of fiber, mineral, and ash.

More particularly, the major components of the dried product according to disclosed embodiments are sugars. In some embodiments, sugars represent about 70% to about 80% of the total by weight. In some embodiments, sugars represent about 60% to about 90% of the total by weight. In some embodiments, sugars represent about 55, 60, 65, 70, 75, 80, 85, 90, or 95% of the total by weight, or a value within a range defined by any two of the aforementioned values. In some embodiments, about 9% to about 25% of the sugars are a 1-4 glucans. In some embodiments, about 5, 10, 15, 20, 25, 30, 35, 40, 45, or 50% of the sugars are a 1-4 glucans, or a value within a range defined by any two of the aforementioned values. In some embodiments, about 8% to about 30% of the sugars are β 1-3 glucans. In some embodiments, about 5, 10, 15, 20, 25, 30, 35, 40, 45, 50, 55, or 60% of the sugars are β 1-3 glucans, or a value within a range defined by any two of the aforementioned values.

The minor components of the dried product include proteins (about 3% to about 4% by weight) and lipid (about 1% to about 3% by weight). In some embodiments, proteins are about 0.5, 1, 1.5, 2, 2.5, 3, 3.5, 4, 4.5, 5, 5.5, 6, 6.5, 7, 7.5, 8% by weight, or a value within a range defined by any two of the aforementioned values. In some embodiments, lipids are about 0.125, 0.25, 0.5, 1, 1.5, 2, 2.5, 3, 3.5, 4, 4.5, 5, 5.5, or 6% by weight, or a value within a range defined by any two of the aforementioned values.

In some embodiments, one or more dried products comprising bioactive fungal compounds are provided. The one or more dried products comprising bioactive fungal compounds are produced according to one or more of the methods disclosed herein. In some embodiments, the dried fungal product is designated K1. In some embodiments, K1 is produced by one or more of the methods provided herein. In some embodiments, K1 is produced by one or more of the methods provided herein using one or more mushroom mycelium cultures. In some embodiments, the one or more mushroom mycelium cultures for producing K1 comprise different strains of Lentinula edodes.

In some embodiments, K1 is produced by one or more of the methods provided herein using a solid substrate comprising hydrated cooked rice, wheat or rye. In some embodiments, K1 is produced by one or more of the methods provided herein comprising an incubation period of about 60 days.

In some embodiments, K1 is characterized by the following properties:
about 81.5% by weight carbohydrates;
about 3.4% by weight protein;
about 0.7% lipid by weight;
about 14.4% by weight of other components; and
immunomodulatory bioactivity.

In some embodiments, K1 is characterized as comprising about 75% to about 85% by weight carbohydrates. In some embodiments, K1 is characterized as comprising about 2.5% to about 5% by weight proteins. In some embodiments, K1 is characterized as comprising about 0.5% to about 1.5% by weight lipids. In some embodiments, other components comprise about 8.5% to about 22% by weight of K1.

In some embodiments, K1 is characterized by HPLC. In some embodiments, K1 is characterized by HPLC of a de-lipidized dried product sample run on an Agilent Hi-Plex Na column. In some embodiments, K1 exhibits three retention time peaks by HPLC on Agilent Hi-Plex Na column. In some embodiments, K1 exhibits three retention time peaks by HPLC on Agilent Hi-Plex Na column at about 10.5, 11.2, and 24.0. In some embodiments, the column is eluted with water at 0.3 ml/min at 85° C. and under a pressure of about 25 bar. In some embodiments, the column eluate is detected by UV at 280 nm.

In some embodiments, the dried product is designated K2. In some embodiments, K2 is produced by one or more of the methods provided herein. In some embodiments, K2 is produced by one or more of the methods provided herein using one or more mushroom mycelium cultures. In some embodiments, the one or more mushroom mycelium cultures for producing K2 comprise a mixture of Coriolus versicolor, Morchella rufobrunnea, and Schizophyllum commune.

In some embodiments, K2 is produced by one or more of the methods provided herein using a solid substrate of hydrated cooked wheat supplemented with soy grits, whole yeast, sucrose and gypsum. In some embodiments, K2 is produced by one or more of the methods provided herein comprising an incubation period of about 35 days to about 45 days.

In some embodiments, K2 is characterized by the following properties:
about 66.8% by weight carbohydrates;
about 4.1% by weight protein;
about 2.7% lipid by weight;
about 26.4% by weight of other components; and
immunomodulatory bioactivity.

In some embodiments, K1 is characterized as comprising about 60% to about 70% by weight carbohydrates. In some embodiments, K1 is characterized as comprising about 3.5% to about 5.5% by weight proteins. In some embodiments, K1 is characterized as comprising about 1.5% to about 4.5% by weight lipids. In some embodiments, other components comprise about 20% to about 35% by weight of K1.

In some embodiments, K2 is characterized by HPLC. In some embodiments, K1 is characterized by HPLC of a de-lipidized dried product sample run on an Agilent Hi-Plex Na column. In some embodiments, K2 exhibits two retention time peaks by HPLC on Agilent Hi-Plex Na column. In some embodiments, K2 exhibits two retention time peaks by HPLC on Agilent Hi-Plex Na column at about 14.3 and 15.2. In some embodiments, the column is eluted with water at 0.3 ml/min at 85° C. and under a pressure of about 25 bar. In some embodiments, the column eluate is detected by UV at 280 nm.

In some embodiments, the one or more dried products comprising bioactive fungal compounds are characterized by the following properties:
60%-90% by weight carbohydrates;
3%-5% by weight protein;
0.1%-3% by weight lipid;
2 or 3 retention time peaks by HPLC; and
immunomodulatory bioactivity.

In some embodiments, the carbohydrates in the dried product comprises polysaccharides. In some embodiments, the polysaccharides comprise α(1-4) glucans and β(1-3) glucans as determined by 1H spectrum. In some embodiments, the polysaccharides comprise α(1-4) glucans and β(1-3) glucans as determined by 13C NMR spectrum. In some embodiments, the polysaccharides comprise α(1-4) glucans and β(1-3) glucans as determined by 1H or 13C NMR spectrum. In some embodiments, the polysaccharides comprise α(1-4) glucans and β(1-3) glucans as determined by 1H and 13C NMR spectrum. In some embodiments, the polysaccharides have a median molecular weight of about 10,000 Da. In some embodiments, the polysaccharides have a median molecular weight of about 7,500 Da to about 12,500 Da. In some embodiments, the polysaccharides have a median molecular weight of about 7,500, 8,000, 8,500, 9,000, 9,500, 10,000, 10,500, 11,000, 11,500, 12,000, or 12,500 Da, or a value within a range defined by any two of the aforementioned values.

In some embodiments, the dried product comprises about 3% to about 5% protein by weight. In some embodiments, the dried product comprises 0.5, 1, 1.5, 2, 2.5, 3, 3.5, 4, 4.5, 5, 5.5, 6, 6.5, 7, 7.5, 8, 8.5, 9, 9.5, or 10% protein by weight, or a value within a range defined by any two of the aforementioned values.

In some embodiments, the dried product comprises about 0.1% to about 3% lipid by weight. In some embodiments, the dried product comprises 0.0125, 0.025, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, or 6% lipid by weight, or a value within a range defined by any two of the aforementioned values.

In some embodiments, the dried product yields two retention time peaks by HPLC of de-lipidized sample on Agilent Hi-Plex Na column. In some embodiments, the dried product yields three retention time peaks by HPLC of de-lipidized sample on Agilent Hi-Plex Na column. In some embodiments, the dried product yields two or three retention time peaks by HPLC of de-lipidized sample on Agilent Hi-Plex Na column.

### Biological Characterization of Fungal Products

In some embodiments, the dried product exhibit biological activities. In some embodiments, the dried product exhibits immunomodulatory bioactivity. Non-limiting examples include anti-viral activity, immune cell activation, induction of cytokines, induction of chemokines, and/or induction of growth factors. In some embodiments, the biological activities of the dried extract are determined. The biological activities of the powdered extract may be determined by any conventional assays such as FACS, ELISA, ELISPOT, Western blotting, immunoassays, cell-based in vitro assays and animal-based in vivo assays, etc.

In some embodiments, the immunomodulatory bioactivity comprises activation of one or more cells selected from NK cells, B-lymphocytes, dendritic cells, stem cells or monocytes. In some embodiments, the immunomodulatory bioactivity comprises at least one of activation of one or more immune cells selected from NK cells, NKT cells, T-lymphocytes, Non-T and Non-NK lymphocytes, or monocytes, activation of pro-inflammatory cytokines/chemokines, activation of anti-inflammatory cytokines/chemokines, and activation of both pro- and anti-inflammatory cytokines/chemokines.

In some embodiments, K1 exhibits biological activities. In some embodiments, K1 exhibits immunomodulatory bioactivity. Non-limiting examples include anti-viral activity, immune cell activation, induction of cytokines, induction of chemokines, and/or induction of growth factors. In some embodiments, K1 exhibits immunomodulatory bioactivity. In some embodiments, the immunomodulatory bioactivity comprises at least one of activation of one or more immune cells selected from NK cells, NKT cells, T-lymphocytes, Non-T and Non-NK lymphocytes, or monocytes, activation of pro-inflammatory cytokines/chemokines, activation of anti-inflammatory cytokines/chemokines, and activation of both pro- and anti-inflammatory cytokines/chemokines.

In some embodiments, K2 exhibits biological activities. In some embodiments, K2 exhibits immunomodulatory bioactivity. Non-limiting examples include anti-viral activity, immune cell activation, induction of cytokines, induction of chemokines, and/or induction of growth factors. In some embodiments, K2 exhibits immunomodulatory bioactivity. In some embodiments, the immunomodulatory bioactivity comprises activation of one or more cells selected from NK cells, B-lymphocytes, dendritic cells, stem cells or monocytes.

In some embodiments, the dried product according to the present disclosure may be useful in preventing and/or treating diseases. Non-limiting examples, include cancer, immune-related diseases such as autoimmune diseases, allergies and inflammation, and infectious diseases, such as influenza, common cold and respiratory illnesses.

### Delivery Routes and Formulations

In some embodiments, the dried fungal extract may be formulated in a form suitable for delivery to a subject having a condition in need of treatment or a condition at risk of development in need of prevention. In some embodiments, the subject is a human. In some embodiments, the subject is a non-human. In some embodiments, the disclosed methods of producing the dried product further comprises formulating the dried product into a form suitable for delivery to a human and a non-human.

In some embodiments, the dried product is formulated in a form suitable for delivery as a dietary supplement, a nutraceutical, a medical food, an animal feedstuff, and/or a nasal spray. In some embodiments, the method further comprises formulating the dried product into a form suitable for delivery via one or more routes of administration.

Non-limiting examples of routes of administration include parenteral, subcutaneous, intravascular injection or infusion, intramuscular injection, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

In some embodiments, the dried product is formulated within one or more suppositories. In some embodiments, the one or more suppositories comprise active ingredients, inactive ingredients, excipients, additives, and/or pharmaceutically acceptable carriers. Non-limiting examples of additives include natural polymer compounds, inorganic salts, binders, lubricants, disintegrants, surfactants, thickeners, coating agents, pH adjusters, antioxidants, flavoring agents, preservatives, and colorants among others. Non-limiting examples of other pharmaceutically acceptable carriers include liquid carriers such as water, alcohol, emulsion, and solid carriers such as gel, powder, and the like.

In some embodiments, the dried product is formulated for intravenous administration with excipients and pharmaceutically acceptable carries including one or more of sodium chloride, dextrose, and sterile water, for example, in the form of aqueous isotonic sterile injection solutions, comprising one or more of antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some embodiments, the dried product is formulated for administration by intravenous infusion such as injection solutions and suspensions prepared from sterile powders, granules, and/or tablets in unit-dose or multi-dose in sealed containers such as ampules and/or vials.

In some embodiments, the dried product is formulated in the form of a dietary supplement tablet or capsule. In some embodiments, the method further comprises formulating the dried product with other components to be used as a nutraceutical or medical food for human consumption or formulated with animal feed material as a substitute for antibiotics. Examples including formulating the dried product with dietary supplements, food additives, nutrients, micronutrients, vitamins, minerals, additional active agents, as well as conventional excipients used in oral delivery formulations.

In some embodiments, the dried product is formulated for oral administration in any dosage form that is suitable for oral ingestion. Non-limiting examples include liquid compositions such as elixir, suspension, syrup, emulsion, ampoule, etc., solid compositions such as gel, gum, drop, powder, granule, pill, sugar-coated tablet, film-coated tablet, capsule, package agent, sustained-release compositions such as gel-coated compositions, multi-coated compositions, localized release compositions, and the like.

In some embodiments, the method further comprises formulating the dried product for nasal administration in any dosage form that is suitable for nasal delivery. Non-limiting examples include nasal spray, nasal drops, metered dose inhalers, aerosols, and the like. Nasal delivery formulations and methods may be useful in treating and/or preventing commono cold, influenza, and allergies.

### EXAMPLES

The following examples are non-limiting.

### Example 1—General Methods of Preparing a Solid Substrate

The solid substrate medium used was hydrated cooked grain. The preferred grain is rice, oats, wheat, rye or millet. The grain was pre-soaked in water and heated to 100° C. for 30 min-60 min. While heating, additional carbohydrates can be added that include glucose, maltose or sucrose along with nitrogen supplements such as soy protein, dried yeast, or peptone. Excess water was removed by draining or decanting and moisten grain is added to autoclavable containers either polypropylene jars or polypropylene bags both of which have filters to allow gas exchange. The filled containers were autoclaved at 121° C. and 15-18 psi for 60 min. The sterilized containers were cooled to room temperature and aseptically inoculated with appropriated strains of fungi. The inoculated jars or bags were subsequently sealed and incubated in the dark for a period between 35 d-60 d at a temperature of preferably 16° C.-21° C. and a RH of 70%.

### Example 2—Cultivation

K1 was produced using a mix of six different cultivars (LE-1 through LE-6) of Lentinula edodes cultivated on solid substrate of rice and wheat for 60 d. K2 was produced using a mixture of Coriolus versicolor, Morchella rufobrunnea, and Schizophyllum commune cultivated on solid substrate of wheat supplemented with soy grits, whole yeast, sucrose and gypsum for a period of 45 d.

### Example 3—Extraction

After cultivation (Example 2), the grain colonized mycelium was removed from the container and dried at 70° C. overnight then ground into a fine powder. The extraction involved heating 500 ml water to between 80° C.-90° C. and adding 50 g of the dried powder. The mixture was covered and heated with continuous agitation for a period of 2 hr, at which time, 2 grams amylase was added to the mixture, and extraction continued for another 1 hr. The slurry was centrifuged at 4,500 g for 10 min to remove debris, the supernatant decanted and freeze dried to obtain the powdered K1 and K2 product. The yield for K1 averaged 10 g/50 g dry weight starting material while K2 yields averaged 15 g/50 g dry weight starting material.

### Example 4—Characterization

| | | | | | |
|---|---|---|---|---|---|
| Total sugar | Protein | Lipid | Other | Range α glucans | Range β glucans |

K1 a slightly pale ochre powder with the following composition:

| | | | | | |
|---|---|---|---|---|---|
| 81.5 g/100 g | 3.4 h/100 g | 0.7 g/100 g | 14.4 g/100 g | 9.9%-25.0% | 10.9%-30.2% |

K2 a tan to light brown powder with the following composition:

| | | | | | |
|---|---|---|---|---|---|
| 66.8 g/100 g | 4.1 g/100 g | 2.7 g/100 g | 26.4 g/100 g | 10.3%-24.2% | 8.3%-21.7% |

Average molecular weight determination of polysaccharide fraction obtained from Sephadex gel filtration chromatography for K1 and K2:
Less than 1,500 Da=13.2%
Between 1,500 Da-5,000 Da=10.3%
Between 5,000 Da-10,000 Da=25.0%
Greater than 10,000 Da=51.5%

Primary sugar as determined by paper chromatography and NMR analysis is glucose for both KI and K2.

Characteristic retention time fingerprint and % composition of de-lipidized sample by high performance liquid chromatography utilizing Agilent Hi-Plex Na column with water elution 0.300 ml/min. at 85° C. and a pressure of 25 bar, with UV detection (at 280 nm).

| Sample | RT | % Area |
|---|---|---|
| K1 | 10.505 | 32.6 |
| | 11.246 | 29.2 |
| | 24.009 | 38.2 |
| K2 | 14.333 | 50.2 |
| | 15.158 | 49.8 |

Major peaks of K1 and K2 as shown for ¹H and ¹³C NMR spectrum:

| Sample | Component | ¹H NMR | ¹³C NMR |
|---|---|---|---|
| K1 | Aliphatic | 0.9-1.0, 1.5, 2.1, 2.4, 2.6-2.7 ppm | |
| | Sugars | 3.2, 3.4-3.6, 3.7-3.9 ppm | 62-65, 70-75, 76-80, 95, 97, 101, 106 ppm |
| K1, K2 | B glucose | 4.6-4.7 ppm | |
| K1, K2 | α glucose | 5.2 ppm | |
| K1, K2 | α 1-4 glucose | 5.4 ppm | |
| K2 | Aliphatic | 1.0, 1.26, 1.5, 1.75, 2-2.25, 2.4, 2.6 ppm | |
| | Sugars | 3.2, 3.4-3.6, 3.7, 3.9 ppm | 61, 64, 70-75, 76-80, 95, 97,101 ppm |
| | Aromatics | 6.55, 6.9, 7.2, 7.4-7.5 ppm | |

### Example 5—Bioactivity

The immunomodulating properties of bioactive compounds of medicinal fungi may include the activation of immune system cells such as T-lymphocytes, NKT killer cells and natural killer cells, as well as activation of the alternative complement pathway, which further stimulates a cascade of immune responses, including the production of cytokines, chemokines and growth factors.

Powdered extracts of K1 and K2 were tested for human immune cell activation and cytokine production. Active hexose correlated compound, AHCC, was included in the test as a comparison. The direct activation of various subsets of immune cell types was conducted by evaluating induction of the CD69 activation marker and flow cytometry. CD69 is a protein expressed on activated white blood cells. It is the earliest inducible cell surface glycoprotein during lymphoid activation resulting in lymphocyte proliferation and cellular signaling. Activation of the CD69 marker is quantified by using a fluorescently labelled monoclonal antibody to the CD69 marker allowing the measurement of cellular activation by increased fluorescence intensity.

In parallel to the testing for immune cell activation, the culture supernatants were saved from cultures and used for testing of secreted biomarkers, i.e. a broad panel of pro- and anti-inflammatory cytokines, anti-viral peptides, and regenerative growth factors, using a Luminex magnetic bead array and the MagPix® multiplexing system. The array measures the levels of IL-1beta, IL-1ra, IL-2, IL-4, IL-5, IL-6, IL-7, IL-8, IL-9, IL-10, IL-12 (p70), IL-13, IL-15, IL-17A, eotaxin, INF-gamma, IP-10, MC-1 (MCAF), MIP-1alpha, MIP-1beta, RANTES, and TNF-alpha.

### Natural Killer (NK) Cells

Following activation, NK cells express CD69 and therefore an increase in this glycoprotein on the surface of NK cells reflects activation. The main downstream effects of CD69 expression by NK cells are associated with increased cytotoxicity. Given this role of CD69 on NK cells, the increase seen in response to some products suggests that these products support innate immunity, particularly that towards virally-infected or transformed (cancer) cells through the sustained activation and increased cytotoxicity of NK cells. In this regard K1 triggered a robust, dose dependent increase in CD69 expression on NK cells, which at the two highest doses exceeded the levels of activation induced by two positive controls, IL-2 and LPS. The increases in CD69 expression were statistically significant for all doses of K1. AHCC did not trigger an increase in CD69 expression on NK cells.

### Natural Killer T (NKT) Cells

CD3+CD56+NKT cells share properties with both NK cells and T cells. Following activation, NKT cells express CD69 and therefore an increase in this glycoprotein on the surface of NKT cells reflects a state of activation. NKT cells have regulatory roles in autoimmunity and work alone as well as in concert with CD4+CD25+ regulatory T cells (Tregs). In this regard K1 triggered a mild dose dependent increase in CD69 expression on NKT cells, which at the highest dose was equal to the levels of activation induced by the positive control LPS. The effect was statistically significant at the lowest dose and highly significant at the three higher doses. Both AHCC and K2 triggered a very mild increase in CD69 expression on NKT cells.

### T Lymphocytes

Activation of T lymphocytes through the T cell receptor (TCR) results in the up-regulation of several proteins including the immediate early activation marker, CD69. Increased expression of CD69 on T cells is associated with an increase in T cell proliferation. In this regard K1 triggered a mild dose dependent increase in CD69 expression on T cells, which at the highest dose was equal to the levels of activation induced by the positive control LPS. The effect was statistically significant at the three lower doses and highly significant at the highest dose. AHCC did not trigger a change in CD69 expression on T cells.

### Non-T Non-NK Lymphocytes-CD69 Expression

The remaining portion of the lymphocyte population includes non-T, non-NK cells. This population includes primarily the B lymphocytes, and includes some dendritic cell types, stem cells, and other rare cell types. In this regard K1 triggered a robust, dose dependent increase in CD69 expression on non-NK non-T cells, which at the higher two doses exceeded the levels of activation induced by the positive controls IL-2 and LPS. The effect was highly significant at all four doses of K1. Both AHCC and K2 triggered a mild change in CD69 expression on non-NK non-T cells, the increase was only observed for the two highest doses and neither was statistically significant.

### Monocyte-CD69 Expression

Monocytes express CD69, and an inflammatory response can be triggered via this cell surface receptor. CD69-mediated inflammatory activation of monocytes triggers unique responses different from CD69-mediated activation of other cell types. The CD69-mediated monocyte response includes production of prostaglandin E2 alpha, 6-keto-prostaglandin F1 alpha, and leukotriene B4, suggesting the activation of cyclooxygenase and lipoxygenase pathways after CD69 stimulation. In this regard both K1 and K2 triggered a robust increase in CD69 expression on monocytes, reaching or exceeding the levels for both positive controls IL-2 and LPS. The effect was highly significant at all four doses of K1 as well as for the two highest doses of K2. AHCC triggered a mild change in CD69 expression on monocytes, the effect was only observed for the highest dose and the increased was not statistically significant.

### Cytokine Induction in Human Peripheral Blood Mononuclear Cell Cultures

The treatment of PBMC cells with K1 led to induction of all immune-activating, pro-inflammatory cytokines tested. AHCC triggered an increase for three cytokines: IL-8, Rantes, MCP-1. For the remaining immune-activating, pro-inflammatory cytokines, AHCC did not cause an increase, but levels were either at or below levels of detection (BLD), or only marginally detectable at one or two doses (indicated by +) (Table 2).

TABLE 2
Comparison of pro-inflammatory cytokines/chemokines

| Cytokines/Chemokines | K1 | K2 | AHCC |
|---|---|---|---|
| IFN-γ | ++ | +++ | (+) |
| IL-1β | ++++ | + | 0 |
| IL-5 | ++ | + | (+) |
| IL-6 | +++ | + | (+) |
| IL-8 | AHS | AHS | +++ |
| IL-12p70 | + | + | BLD |
| IL-13 | + | (+) | BLD |
| IL-I7A | ++ | + | (+) |
| Eotaxin | + | + | (+) |
| IP-10 | ++ | + | (+) |
| MCP-1 | ++ | ++++ | + |
| MIP-1α | AHS | ++++ | (+) |
| MIP-1β | +++++ | +++ | (+) |
| RANTES | +++ | +++ | ++ |
| TNF-α | +++++ | +++ | (+) |

BLD: Below Levels of Detection, i.e. the levels were below lowest dose of the standard curve.
AHS: Above highest dose of standard, i.e. the levels exceeded the highest dose that could be measured with accuracy.

### Anti-Inflammatory Cytokines/Chemokines

Both K1 and AHCC triggered increases in the anti-inflammatory cytokine interleukin-1 receptor antagonist. K1 triggered a highly robust increase, AHCC showed a mild increase. K1 also triggered a robust increase in the anti-inflammatory cytokine IL-10, whereas AHCC did not induce this cytokine to a detectable level (Table 3).

TABLE 3
Comparison of anti-inflammatory cytokines

| Cytokines | K1 | K2 | AHCC |
|---|---|---|---|
| IL-1ra | ++++ | +++ | + |
| IL-10 | +++ | + | (+) |

Cytokines/Chemokines with both Pro- and Anti-Inflammatory Properties

K1 induced a strong response for all five regulating cytokines, while AHCC only induced IL-9 to detectable level, and only marginally induced IL-2 (Table 4).

TABLE 4
Comparison of pro- and anti-inflammatory cytokines/chemokines

| Cytokines/Chemokines | K1 | K2 | AHCC |
|---|---|---|---|
| IL-2 | ++ | + | (+) |
| IL-4 | + | + | BLD |
| IL-7 | + | + | BLD |
| IL-9 | ++ | ++ | + |
| IL-15 | ++ | ++ | BLD |

BLD: Below Levels of Detection, i.e. the levels were below lowest dose of the standard curve.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of any one of the aspects is applicable to all aspects and embodiments identified herein. Moreover, any of the features of an embodiment any one of the aspects is independently combinable, partly or wholly with other embodiments described herein in any way, e.g., one, two, or three or more embodiments may be combinable in whole or in part. Further, any of the features of an embodiment of any one of the aspects may be made optional to other aspects or embodiments.

What is claimed is:

1. A solid-state fermentation method for producing a dried product comprising bioactive fungal compounds, the method comprising:
inoculating a solid substrate with one or more mushroom mycelium cultures;
incubating the inoculated solid substrate for a period of about 10-100 days under conditions selected to promote colonization and enzymatic hydrolysis of the solid substrate to produce a hydrolysate;
extracting the hydrolysate in water at a temperature of about 60° C.-100° C. with agitation;
treating the extract with amylase;
clarifying the extract to remove undigested material; and
drying the clarified extract to yield a dried product comprising bioactive fungal compounds.

2. The method of claim 1, wherein the solid substrate comprises a hydrated cooked grain.

3. The method of claim 2, wherein the hydrated cooked grain comprises one or more grains selected from rice, wheat, millet, oats or rye, and wherein the one or more grains are hydrated in water and cooked for about 10-90 minutes at a temperature of about 70° C.-120° C.

4. The method of claim 3, wherein the grains are cooked for about 30-60 minutes at 100° C.

5. The method of any one of claims 1-4, wherein the solid substrate further comprises one or more of a carbohydrate, a nitrogen source and a mineral supplement.

6. The method of claim 5, wherein the carbohydrate is selected from glucose, sucrose, maltose, malt extract, corn steep liquor or combinations thereof.

7. The method of claim 5, wherein the nitrogen source is selected from peptone, soybean powder, soy grits, whole yeast, yeast extract or combinations thereof.

8. The method of claim 5, wherein the mineral supplement is selected from phosphates, calcium, magnesium, iron, sulfates or combinations thereof.

9. The method of any one of claims 1-8, wherein the solid substrate is sterilized before inoculation.

10. The method of claim 9, wherein the solid substrate is sterilized by autoclaving for about 30-120 minutes at a temperature of about 100° C.-200° C. at a pressure of about 10-30 psi.

11. The method of claim 10, wherein the solid substrate is sterilized by autoclaving for about 60 minutes at a temperature of about 121° C. at a pressure of about 15-18 psi.

12. The method of any one of claims 1-11, wherein the one or more mushroom mycelium cultures comprise a fungal class selected from Ascomycetes or Basidiomycetes.

13. The method of claim 12, where the one or more mushroom mycelium cultures are Morchella rufobrunnea from the Ascomycetes class.

14. The method of claim 12, where the one or more mushroom mycelium cultures are from the Basidiomycetes class, selected from Coriolus versicolor, Lentinula edodes or Schizophyllum commune.

15. The method of any one of claims 1-14, wherein the incubation conditions comprise incubating in the dark for 30-60 days.

16. The method of any one of claims 1-15, wherein the incubation conditions comprise incubating at a temperature of about 10° C.-40° C.

17. The method of claim 16, wherein the incubation temperature is about 16° C.-21° C.

18. The method of any one of claims 1-17, wherein the incubation conditions comprise incubating at a relative humidity of about 50%-100%

19. The method of claim 18, wherein the incubation relative humidity is about 70%.

20. The method of any one of claims 1-19, wherein drying comprises freeze-drying (lyophilization), spray-drying, heating, drying under nitrogen and/or combinations thereof.

21. The method of any one of claims 1-20, further comprising formulating the dried product into a form suitable for delivery as a dietary supplement, nutraceutical, medical food or animal feedstuff.

22. A dried product comprising bioactive fungal compounds produced by the method of claim 1, wherein the one or more mushroom mycelium cultures comprise different strains of Lentinula edodes; the solid substrate comprises of hydrated cooked rice, wheat or rye; and the incubation period comprises 60 days; and wherein the dried product is characterized by the following properties:
about 81.5% by weight carbohydrates;
about 3.4% by weight protein;
about 0.7% lipid by weight;
about 14.4% by weight of other components; and
immunomodulatory bioactivity.

23. The dried product of claim 22, wherein the dried product is further characterized by exhibiting 3 retention time peaks at about 10.5, 11.2, and 24.0 by HPLC of a de-lipidized dried product sample run on an Agilent Hi-Plex Na column eluted with water at 0.3 ml/min at 85° C. and a pressure of 25 bar.

24. The dried product of claim 22, wherein the immunomodulatory bioactivity comprises at least one of the following:
activation of one or more immune cells selected from NK cells, NKT cells, T-lymphocytes, Non-T and Non-NK lymphocytes, or monocytes; and
activation of pro-inflammatory cytokines/chemokines, or anti-inflammatory cytokines/chemokines, or both pro- and anti-inflammatory cytokines/chemokines.

25. A dried product comprising bioactive fungal compounds produced by the method of claim 1, wherein the one or more mushroom mycelium cultures comprise a mixture of Coriolus versicolor, Morchella rufobrunnea, and Schizophyllum commune; the solid substrate comprises supplemented (sucrose, soy grits, whole yeast and gypsum) hydrated cooked wheat; and the incubation period comprises 35-45 days; and wherein the dried product is characterized by the following properties:
about 66.8% by weight carbohydrates;
about 4.1% by weight protein;
about 2.7% lipid by weight;
about 26.4% by weight of other components; and
immunomodulatory bioactivity.

26. The dried product of claim 25, wherein the dried product is further characterized by exhibiting 2 retention time peaks at about 14.3 and 15.2 by HPLC of a de-lipidized dried product sample run on an Agilent Hi-Plex Na column eluted with water at 0.3 ml/min at 85° C. and a pressure of 25 bar.

27. The dried product of claim 25, wherein the immunomodulatory bioactivity comprises activation of one or more cells selected from NK cells, B-lymphocytes, dendritic cells, stem cells or monocytes.

28. A dried product comprising bioactive fungal compounds, wherein the dried product is characterized by the following properties:
60%-90% by weight carbohydrates, comprising polysaccharides including α(1-4) glucans and β(1-3) glucans as determined by ¹H and ¹³C NMR spectrum, the polysaccharides having a median molecular weight of about 10,000 Da;
3%-5% by weight protein;
0.1%-3% by weight lipid;
2 or 3 retention time peaks by HPLC of de-lipidized sample on Agilent Hi-Plex Na column; and
immunomodulatory bioactivity.

29. The dried product of any one of claims 22-28, wherein the dried product is in a form suitable for delivery as a dietary supplement, nutraceutical, medical food or animal feedstuff.
